(19) United States
(12) Patent Application Publication
(10) Pub. No.: US 2025/0345547 A1
(43) Pub. Date: Nov. 13, 2025

(54) Medical Mask

(71) Applicant: Upstream MedTech LLC, Far Hills, NJ (US)

(72) Inventor: Kathryn Gutkin, Lincroft, NJ (US)

(21) Appl. No.: 18/657,213

(22) Filed: May 7, 2024

(51) Int. Cl. A61M 16/06

(52) U.S. Cl. CPC A61M 16/0616; A61M 2202/0208

(57) ABSTRACT

Described is a medical mask comprising a body having an upper portion and a lower portion, the upper portion comprising an aperture adapted to receive a nasal mask, the lower portion comprising a mouth covering with a temporarily sealed opening which, when unsealed, is adapted to receive a medical device into an interior of the lower portion; a gas inlet port disposed on the lower portion, the gas inlet port adapted to receive an external gas supply for introduction of gas into the interior of the lower portion; and a gas sampling port disposed on the lower portion, the gas sampling port adapted to receive an external gas sampling device to sample gas from the interior of the lower portion.

### BACKGROUND OF THE INVENTION

In oral endoscopic procedures, a physician inserts an endoscope through a patient's mouth and into the patient's upper gastrointestinal tract. The endoscope is used to visualize the patient's esophagus, stomach and/or small intestine. The endoscope may accommodate tools for performing, for example, tissue biopsies, obstruction removal, fluid/air injections, cauterizations, etc.

It is becoming increasingly common to perform endoscopies under sedation, to minimize patient discomfort and anxiety, and provide more efficient and better quality examination. To reduce the risk of respiratory depression, airway obstruction, and/or hypoxemia, the standard of care is to administer supplementary oxygen using a nasal cannula. The nasal cannula may provide oxygen and measure end-tidal CO₂, allowing physicians and other clinical staff to monitor the patient's ventilation in real-time.

Due to a relatively low amount of oxygen that can be delivered through the nasal cannula and to assist patients with respiratory issues (e.g., sleep apnea, supplemental oxygen, smoking history, etc.) and other medical conditions (e.g., obesity, poor circulation, etc.), two alternatives have been proposed.

One alternative is a nasal continuous airway positive pressure (CPAP) mask (e.g., SuperNO₂ VAR). The nasal CPAP mask creates a seal around the patient's nose to administer oxygen, air and anesthetic gas. The nasal CPAP mask also includes a capnography port for measuring end-tidal CO₂. Use of the nasal CPAP mask provides supplementary oxygen, while allowing the physician access to the patient's mouth. While the nasal CPAP mask may permit delivery of more supplementary oxygen than the nasal cannula, there is no barrier over the patient's mouth to protect clinical staff from exhaled pathogens (e.g., flu, COVID-19, etc.), and there is no means by which to administer supplemental oxygen and/or measure oral end-tidal CO₂ if, for example, the patient breathes through his/her mouth, and/or has a nasal condition (e.g., polyps, inflammation, etc.) which reduce the efficacy of the nasally-delivered oxygen.

A second alternative is an oxygen mask with endoscopic valves (e.g., a Procedural Oxygen Mask® (POM®)). The oxygen mask covers the patient's nose and mouth, and provides valves adjacent to the nose and mouth for permitting entry and exit of bronchoscopes/endoscopes. The oxygen mask with nasal and oral endoscopic valves allows for the delivery of supplementary oxygen, provides a covering over the patient's nose and mouth, and allows access to the patient's nose and mouth for scopes. However, the oxygen mask is not a closed-system like the nasal CPAP mask and thus delivers significantly less oxygen to the patient, especially when one or more of the valves are punctured to permit access to the nasal or oral cavity.

Due to the designs of the nasal cannula, the nasal CPAP mask, and the oxygen mask with endoscopic valves, none of these devices can be used together. Thus, there exists a significant unmet medical need to provide a device that maximizes a fraction of inspired oxygen (FiO2) to a patient during an endoscopic procedure. Such a need is particularly acute for patients that, for example, require daily supplemental oxygen, patients with respiratory issues (e.g., obstructive sleep apnea, smoking history, nasal polyps, etc.), patients that breathe through their mouths, and obese and/or overweight patients.

### SUMMARY OF THE INVENTION

Illustrative embodiments of the invention will hereinafter be described in conjunction with the appended drawings provided to illustrate, but not limit, the invention.

A medical mask according to an exemplary embodiment of the present invention comprises a body having an upper portion and a lower portion, the upper portion comprising an aperture adapted to receive a nasal mask, the lower portion comprising a mouth covering with a temporarily sealed opening which, when unsealed, is adapted to receive a medical device into an interior of the lower portion; a gas inlet port disposed on the lower portion, the gas inlet port adapted to receive an external gas supply for introduction of gas into the interior of the lower portion; and a gas sampling port disposed on the lower portion, the gas sampling port adapted to receive an external gas sampling device to sample gas from the interior of the lower portion.

### BRIEF DESCRIPTION OF THE DRAWINGS

FIG. 1 shows an exemplary embodiment of a medical mask in a perspective view according to the present invention.

FIG. 2 shows an exemplary embodiment of a medical mask in a front view according to the present invention.

FIG. 3 shows an exemplary embodiment of a medical mask fitted on a patient according to the present invention.

FIG. 4 shows another exemplary embodiment of a medical mask in a front view according to the present invention.

FIG. 5 shows another exemplary embodiment of a medical mask in a front view according to the present invention.

### DETAILED DESCRIPTION

The following detailed description is merely exemplary in nature and is not intended to limit the invention, but to describe exemplary embodiments of the present invention. Exemplary embodiments of the present invention are directed to a medical mask for providing gas (e.g., oxygen, anesthesia, etc.) to a patient during procedures in which a medical device such as, for example, a scope, a probe, a tube or other instrument is inserted into the patient's mouth. The medical mask according to the exemplary embodiments of the present invention may be made from plastic, rubber, silicone and/or any combination thereof. The medical mask may also be latex free.

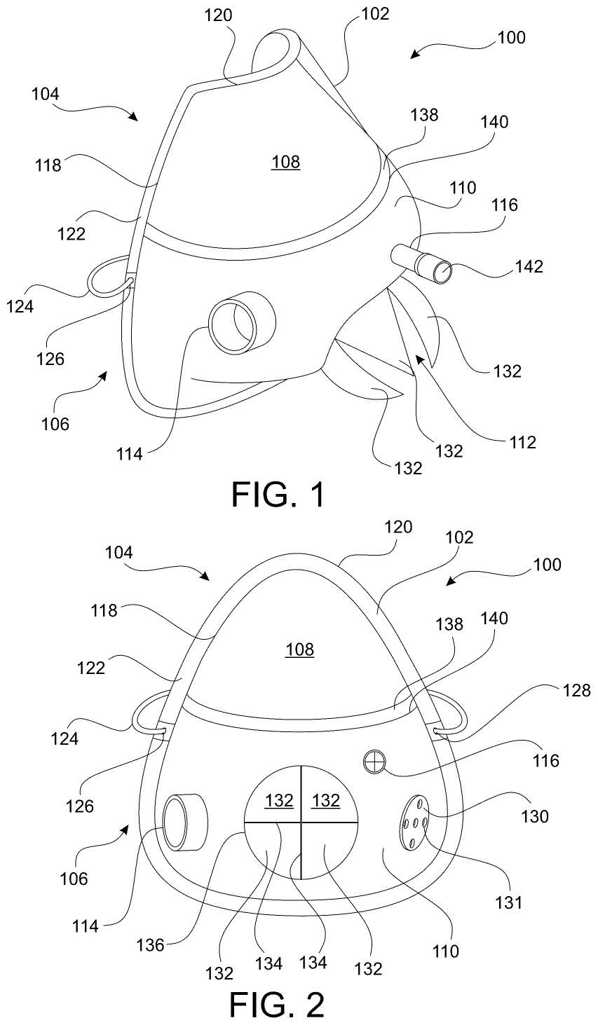

FIG. 1 shows an exemplary embodiment of a medical mask 100 according to the present invention. The medical mask 100 has a body 102 with an upper portion 104 and a lower portion 106. The upper portion 104 comprises an aperture 108 adapted to receive a nasal mask. For example, if a patient is wearing a nasal CPAP mask, the nasal CPAP mask may fit within the aperture 108. In an exemplary embodiment, the aperture 108 is substantially triangular.

The lower portion 106 of the medical mask 100 includes a mouth covering 110 that is sized and shaped to fit over a mouth of the patient. The mouth covering 110 includes a temporarily sealed opening 112. For example, when the medical mask 110 is manufactured, the opening 112 may be sealed. As will be described further below, during a procedure such as an endoscopy, the opening 112 may be unsealed for permitting an endoscope to access an interior of the lower portion 110. Once unsealed, the opening 112 may not be resealed, except through a fixation device (e.g., tape, clip, etc.).

The lower portion 106 of the medical mask 100 includes a gas inlet port 114. The gas inlet port 114 is adapted to receive an external gas supply for introduction of gas into the interior of the lower portion 106. For example, the gas inlet port 114 may be sized and shaped to receive a fitting that permits an external gas supply to be connected thereto, for supplying gas into the interior of the lower portion 106. For example, during an endoscopy, an oxygen source may be connected to the fitting to flow oxygen into the interior of the lower portion 106. The oxygen source may be from an oxygen supply line in an endoscopy suite and/or procedure room, and/or from an anesthesia machine. The gas inlet port 114 may also accommodate a coupling for a ventilation bag, in situations in which manual ventilation is implemented.

The lower portion 106 of the medical mask 100 includes a gas sampling port 116. The gas sampling port 116 may be adapted to receive an external gas sampling device to sample gas from the interior of the lower portion 106. In this manner, the gas sampling port 116 may include a Luer lock, a threaded portion or any other means for mechanically connecting with an external gas sampling device. For example, during an endoscopy, the gas sampling port 116 may allow clinical staff to monitor end-tidal CO₂ being expelled orally by the patient. In an exemplary embodiment, the gas sampling port 116 may be disposed adjacent a top of the opening 112. A removable cap 142 may be disposed over an opening of the gas sampling port 116.

The medical mask 100 comprises a rim 118 extending around the upper portion 104 and the lower portion 106. The rim 118 may encircle the body 102 of the medical mask 100. In an exemplary embodiment, the rim 118 includes an arch 120 formed at a top of the upper portion 104. The arch 120 may be sized and shaped to fit around a bridge of the patient's nose and/or the nasal CPAP mask. When the patient is wearing the nasal CPAP mask, the arch 120 may at least partially engage or rest upon the nasal CPAP mask, which may align the medical mask 100 with the nasal CPAP mask and prevent relative horizontal movement of the medical mask 100 across the patient's face. In an exemplary embodiment, the rim 118 may include a flange 122 that extends radially outward from the rim 118. The flange 122 may be disposed along the entirety of the rim 118 or a portion thereof. When the medical mask 100 is placed on a patient's face, the flange 122 may rest upon the patient's face and provide a seal around at least the lower portion 106. In another exemplary embodiment, an inflatable or pre-inflated cushion may be disposed on the flange 122, at least partially, or in place of the flange 122, at least partially.

When the medical mask 100 is being fitted on a patient, the rim 118 around the upper portion 104 may be pulled radially outward to accommodate a nasal CPAP mask. Due to material properties (e.g., shape memory) of the medical mask 100, the rim 118 may return to its original orientation and shape.

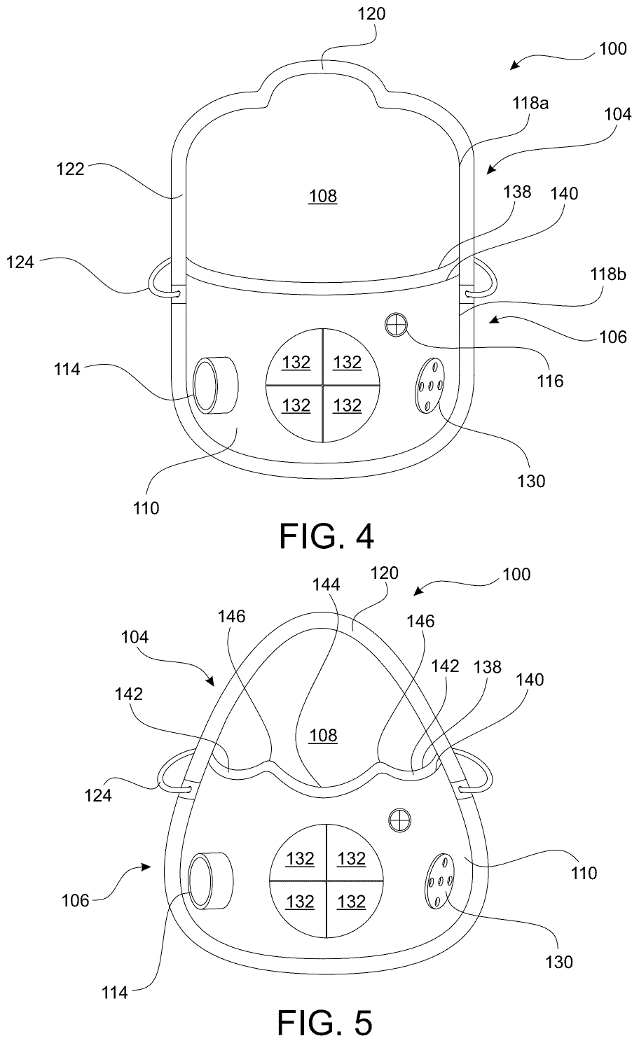

In another exemplary embodiment shown in FIG. 4, the rim 118 includes a top portion 118a around the upper portion 104 that is substantially equal in width to a bottom portion 118b of the rim 118 around the lower portion 106. In this embodiment, the rim 118 may be sufficiently sized such that aperture 108 fully accommodates a nasal CPAP mask, and the rim 118 need not be pulled or distended while fitting the medical mask 100 over a nasal CPAP mask. In yet another exemplary embodiment, the width of the top portion 118a may be greater than the width of the bottom portion 118b. In this exemplary embodiment, the rim 118 (and the aperture 108) may be shaped like an upside-down triangle.

Referring back to FIG. 1, the medical mask 100 may have a strap 124 coupled to the body 102 that may be used to encircle a head of the patient. In one exemplary embodiment, the medical mask 100 may have a first strap coupling 126 and a second strap coupling 128 formed in rim 118 or the flange 122. The first and second strap couplings 126, 128 may be holes cut into the flange 122 which the strap 124 can be tied or knotted on. The first and second strap couplings 126, 128 may be formed in opposite sides of the flange 122 around the lower portion 106. In another exemplary embodiment, the medical mask 100 may have one or more projections (or be fitted with a separate device with such projections) adapted to engage holes in the strap 124, to secure the mask 100 to the patient.

In one exemplary embodiment, the first and/or second strap couplings 126, 128 may include a quick-release mechanism. For example, if a patient has respiratory distress, it may be necessary to intubate. In such a case, clinical staff would need to remove the medical mask 100. The quick-release mechanism could be, for example, a cut through the flange 122 into the hole in the strap couplings 126, 128. Thus, clinical staff could pull the strap 124 out of the hole and through the cut to release the strap 124 and remove the medical mask 100. Those of skill in the art will understand that various other couplings may be utilized to permanently or releasably secure the strap 124 to the mask 100 including, but not limited to, buckles, clips, etc.

As shown in FIG. 2, a one-way valve 130 may be disposed on the lower portion 106. In an exemplary embodiment, the one-way valve 130 may include at least one hole 131 allowing gas to pass outwardly from the interior of the lower portion 106. The one-way valve 130 may include a cover (not shown) tethered to the lower portion 106 and disposed over the at least one hole 131. In this exemplary embodiment, as a patient exhales, the exhaled gas may temporarily displace the cover, allowing the exhaled gas to pass outwardly from the interior of the lower portion 106. After exhalation, or when the patient is inhaling, the cover may return to its position covering the at least one hole 131 on the one-way valve 130.

In an exemplary embodiment, the one-way valve 130 and the gas inlet port 114 may be disposed on the lower portion 106 on opposite sides of the opening 112. In another exemplary embodiment, the one-way valve 130 and the gas inlet port 114 may be disposed on the same side of the opening 112. This may be beneficial when, for example, the patient is disposed on his/her side during a procedure.

In an exemplary embodiment, the temporarily sealed opening 112 comprises at least two flaps 132. Each flap 132 may have a first edge 134 temporarily sealed to a first edge 134 of an adjacent flap 132. Each flap 132 may have a second edge 136 permanently coupled to an edge of the opening 112. As shown in FIG. 2, the first edges 134 of the flaps 132 are temporarily sealed to each other and the opening 112 is covered. In this position, gas supplied to the interior of the lower portion 106 may be substantially maintained around the patient's mouth. As shown in FIG. 1, the first edges 134 of the flaps 132 are unsealed and bent or folded outward, and the opening 112 is uncovered. In this position (or when the flaps are bent or folded inward), the second edge 136 of the flaps 132 may act as a hinge, allowing a physician to insert a medical device (e.g., endoscope) through the opening 112 and into the patient's mouth. In an exemplary embodiment, the hinge may bias the flaps 132 towards the closed or unfolded positions, covering the opening 112 as shown in in FIG. 2. In an embodiment in which the second edge 136 is a biased hinge, the flaps 132 may maintain contact with the medical device disposed through the opening 112 to limit gas outflow from the interior of the lower portion 106 and mitigate the effects of expulsion of aerosolized pathogens if, for example, the patient coughs during the procedure. In an exemplary embodiment, once the first edges 134 of the flaps 132 are unsealed from each other, the first edges 134 cannot be resealed.

FIGS. 1 and 2 show an exemplary embodiment of the medical mask 100 in which the opening 112 is covered by four quarter-circular flaps 132. Those of skill in the art will understand that embodiments of the present invention may encompass any configuration of at least two flaps 132. For example, two semi-circular flaps may be disposed over the opening 112, or three one-third flaps may be disposed over the opening 112. And, while the exemplary embodiment is described with respect to the second edge 136 of the flap 132 being permanently coupled to the lower portion 106, in other exemplary embodiments, the second edge 136 may be temporarily sealed to the edge of the opening 112, such that the flaps 132 may be completely removed from the opening 112.

Referring to the exemplary embodiment of the medical mask 100 shown in FIG. 1, a top 140 of the lower portion 106 may have a flange 138 at least partially formed thereon and projecting radially inward, toward the patient's face. The flange 138 may further prevent gas delivered through the gas inlet port 114 from escaping the interior of the lower portion 106. The flange 138 may extend, for example, at least partially under a patient's nose and/or the nasal CPAP mask, or to nearly or fully abutting a patient skin. The flange 138 may be formed along the full top 140 of the lower portion 106, or along portions thereof.

In another exemplary embodiment shown in FIG. 5, the flange 138 may have two lateral portions 142 and a medial portion 144 therebetween. In another exemplary embodiment, the medial portion 144 may be removed to create a space to accommodate the nasal CPAP mask. As shown in FIG. 5, projections 146 may be formed at interfaces between the lateral portions 142 and the medial portion 144. The projections 146 may extend upward towards the arch 120. When the mask 100 is fit onto a patient wearing a nasal CPAP mask, the projections 146 may abut sides of the nasal CPAP mask to align the mask 100 with the nasal CPAP mask and prevent lateral movement of the mask 100 relative to nasal CPAP mask. In an exemplary embodiment, the flange 138, or any of the lateral portions 142 and/or the medial portion 144, may be concave. In this exemplary embodiment, the shape of the flange 138 may accommodate the nasal CPAP mask.

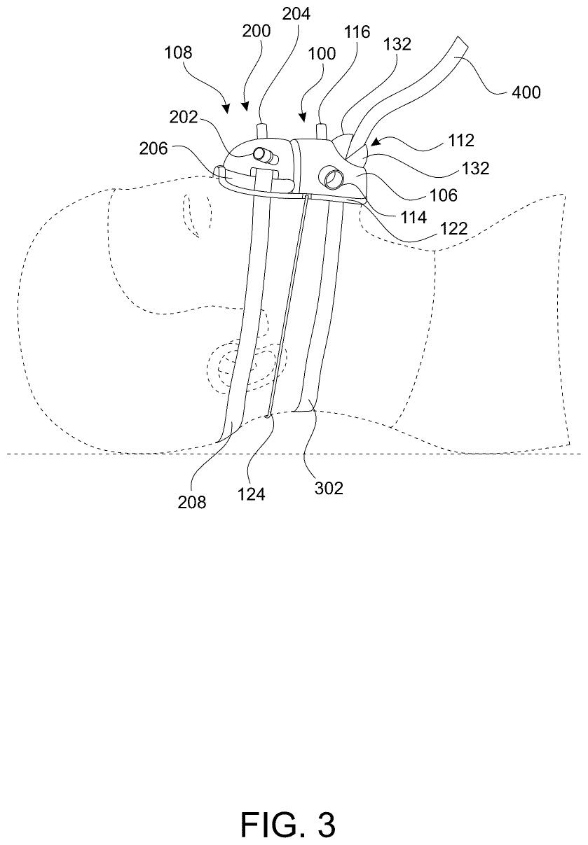

FIG. 3 shows a medical mask 100 according to an exemplary embodiment of the present invention disposed on a patient. While FIG. 3 will be described with respect to an endoscopic procedure, those of skill in the art will understand that the medical mask 100 according to the present invention may be used in other pre-and post-procedure situations (e.g., pre-and post-oxygenation), biopsies, drug delivery, etc.

Prior to an endoscopic procedure, the patient may be fitted with a nasal CPAP mask 200. The nasal CPAP mask 200 may be packaged by itself or with the medical mask 100. Both the nasal CPAP mask 200 and the medical mask 100 may be manufactured in different sizes to accommodate, for example, adult and pediatric patients. The nasal CPAP mask 200 may have a gas inlet port 202 and a gas sampling port 204. The nasal CPAP mask 200 may have a membrane 206 disposed around a perimeter of the nasal CPAP mask 200 to create a sealed environment for the delivery of gas to the patient's nose. The nasal CPAP mask 200 is fitted on the patient's nose and secured by a strap 208.

After the patient is fitted with the nasal CPAP mask 200, a bite block (not shown) may be inserted into the patient's mouth. The bite block includes a strap 302 to fit around the patient's head. The bite block includes an opening to receive the endoscope, while preventing the patient from biting the scope and protecting the patient's teeth.

When the nasal CPAP mask 200 and the bite block have been fitted on the patient, the medical mask 100 may be fitted on the patient using the strap 124. The aperture 108 of the medical mask 100 accommodates the nasal CPAP mask 200, and the arch 120 may abut a portion of the nasal CPAP mask 200. The flange 122 may abut the patient's face. When the medical mask 100 is initially deployed on the patient, the first edges 134 of the flaps 132 are temporarily sealed, and the opening 112 is covered.

During the procedure, gas may be flowed into the nasal CPAP mask 200 through the gas inlet port 202 and flowed into the medical mask 100 through the gas inlet port 114, either alternately or in parallel. For example, the gas inlet port 202 may be coupled to an oxygen supply infrastructure in the room, and the gas inlet port 114 may be coupled to an oxygen supply on an anesthesia machine, or vice-versa. End-tidal CO₂ in the nasal CPAP mask 100 may be monitored via the gas sampling port 202, and end-tidal CO₂ in the medical mask 100 may be monitored via the gas sampling port 116. Nasal and oral end-tidal CO₂ measurement capability allows clinical staff to monitor the patient's ventilation in real-time, preventing emergency intubation. This further allows clinical staff to have objective data regarding the patient's ventilation and oxygen saturation, should any review of the procedure become necessary (e.g., for compliance, litigation, etc.).

An endoscope 400 may be introduced into the interior of the lower portion 106 of the medical mask 100 via the opening 112. The seal on the first edges 134 of the flaps 132 may be broken by a clinical staff member (e.g., using a finger to push the flaps 132 until the first edges 134 separate) or by force from the endoscope 400. After the seal on the first edges 134 of the flaps 132 is broken, a distal end of the endoscope 400 may be pushed through the flaps 132, flexing them into an interior of the lower portion 106, and allowing the endoscope 400 to enter the patient's mouth.

When the endoscope 400 is removed from the patient's mouth and out of the lower portion 106 of the medical mask 100, the flaps 132 may return to their original position, e.g., via the hinged bias of the second edge 136 of the flaps. Preferably, a biasing force of the second edge 136 prevent rotation of the flaps 132 outward due to the patient's breathing and/or coughing. Accordingly, the flaps 132 may be closed over the opening 112, limiting escape of oxygen supplied via the gas inlet port 114. This will allow the patient to continue receiving oxygen via the nasal CPAP mask 200 and/or the medical mask 100 post-operatively.

Since many modifications, variations, and changes in detail, size and shape can be made to the exemplary embodiments of the present invention, it is intended that all matters in the foregoing description and shown in the accompanying drawings be interpreted as illustrative and not in a limiting sense. Furthermore, it is understood that any of the features presented in the exemplary embodiments may be integrated into any of the other exemplary embodiments.

What is claimed is:

1. A medical mask, comprising:
a body having an upper portion and a lower portion, the upper portion comprising an aperture adapted to receive a nasal mask, the lower portion comprising a mouth covering with a temporarily sealed opening which, when unsealed, is adapted to receive a medical device into an interior of the lower portion;
a gas inlet port disposed on the lower portion, the gas inlet port adapted to receive an external gas supply for introduction of gas into the interior of the lower portion; and
a gas sampling port disposed on the lower portion, the gas sampling port adapted to receive an external gas sampling device to sample gas from the interior of the lower portion.

2. The medical mask according to claim 1, wherein the body comprises a rim extending around the upper portion and the lower portion.

3. The medical mask according to claim 2, wherein the rim comprises an arch on the upper portion.

4. The medical mask according to claim 3, wherein the arch is adapted to abut the nasal mask.

5. The medical mask according to claim 2, further comprising a flange extending radially from the rim.

6. The medical mask according to claim 2, wherein the rim includes a top portion around the upper portion and a bottom portion around the lower portion, wherein the top portion has a width equal to or greater than the bottom portion.

7. The medical mask according to claim 1, further comprising a strap having a first end coupled to a first strap coupling on the lower portion and a second end coupled to a second strap coupling on the lower portion, wherein the first strap coupling and the second strap coupling are on opposite sides of the lower portion.

8. The medical mask according to claim 7, wherein at least one of the first strap coupling and the second strap coupling comprises a quick-release mechanism.

9. The medical mask according to claim 1, further comprising a one-way valve disposed on the lower portion.

10. The medical mask according to claim 9, wherein the one-way valve and the gas inlet port are disposed on the lower portion on opposite sides of the temporarily sealed opening.

11. The medical mask according to claim 1, wherein the gas sampling port is disposed adjacent a top of the temporarily sealed opening.

12. The medical mask according to claim 1, wherein the temporarily sealed opening comprises at least two flaps, wherein each of the at least two flaps has a first edge temporarily sealed to the other of the at least two flaps and a second edge permanently coupled to an edge of the opening.

13. The medical mask according to claim 12, wherein the at least two flaps, when unsealed from each other, cannot be resealed to each other.

14. The medical mask according to claim 13, wherein the at least two flaps, when unsealed from each other can fold at least one of inwardly and outwardly.

15. The medical mask according to claim 12, wherein the second edge is a hinge biasing the at least two flaps in an unfolded position.

16. The medical mask according to claim 1, wherein a top of the lower portion has a flange projecting radially inward.

17. The medical mask according to claim 16, wherein the flange substantially covers the top of the lower portion.

18. The medical mask according to claim 16, wherein at least a portion of the flange is concave.

19. The medical mask according to claim 16, wherein the flange includes at least one projection adapted to abut the nasal mask.

20. The medical mask according to claim 1, wherein the gas sampling port includes at least one of a Luer lock and a threaded portion.
